(19) United States
(12) Patent Application Publication
(10) Pub. No.: US 2005/0183729 A1
(43) Pub. Date: Aug. 25, 2005

(54) Dilational device having a reinforced balloon catheter

(76) Inventor: Frank J. Fischer JR., Bloomington, IN (US)

Correspondence Address: BRINKS HOFER GILSON & LIONE/INDY/COOK, ONE INDIANA SQUARE, SUITE 1600, INDIANAPOLIS, IN, 46204-2033, US

(21) Appl. No.: 11/044,773

(22) Filed: Jan. 27, 2005

Related U.S. Application Data
(60) Provisional application No. 60/539,750, filed on Jan. 28, 2004.

(51) Int. Cl. A62B 9/06; A61M 16/00

(52) U.S. Cl. 128/207.29; 128/207.14

(57) ABSTRACT

A device for providing an ostomy through the wall of the trachea. The device includes a dilator tube having a distal end, and a reinforced balloon catheter carried by the dilator tube. The reinforced balloon catheter includes an inflatable balloon extending from the distal end of the dilator tube. The dilator tube and the reinforced balloon catheter are advanceable along a wire guide percutaneously positionable across the tracheal wall, and the reinforced balloon is adapted to atraumatically dilate a portion of the tracheal wall to form an ostomy in the tracheal wall upon inflation of the balloon. The balloon is reinforced with a metal or metal alloy to inhibit puncture by sharp portions of the trachea. The balloon is adapted to have a curved orientation upon inflation to diminish stress on the posterior wall of the trachea.

### RELATED APPLICATION

The present patent document claims the benefit of the filing date under 35 U.S.C. §119(e) of Provisional U.S. Patent Application Ser. No. 60/539,750, filed Jan. 28, 2004, which is hereby incorporated by reference.

### BACKGROUND

1. Technical Field

This invention relates generally to medical devices used for percutaneously accessing a patient's air passageway and atraumatically dilating an opening thereto, and in particular, to a device for use in forming an ostomy in a tracheal wall comprising a reinforced balloon catheter.

2. Background Information

The establishment of an adequate air passageway is the first critical step in maintaining the ability of a seriously ill or injured patient to breathe, or in performing resuscitation on a patient unable to breathe. Endotracheal intubation (the placement of a tube through the nostrils or mouth and into the trachea itself) is a preferred method for establishing an air passageway when the trachea, nostrils and/or mouth are free of obstruction. When an obstruction is present, however, endotracheal intubation is not possible, so an alternative passageway for airflow must be established.

The most direct way to provide an air passageway under these circumstances is to form an ostomy or opening in the tracheal wall, and once formed, to keep the ostomy open by inserting a tracheal tube into it. Conventional tracheal tubes generally include an open distal aperture and a circumferential inflatable cuff. The cuff provides a seal between the tracheal wall and the tracheal tube at a location cranial to the distal aperture to prevent the intrusion of blood, tissue or foreign matter into the lower trachea, bronchi and lungs.

Several methods and devices are known for forming or enlarging an ostomy in a tracheal wall. For example, tracheostomy and cricothyrotomy procedures have been performed by using a scalpel to make an incision in the neck. Such procedures often entail a high degree of surgical skill to perform successfully, particularly since it is vital to locate and avoid unintentional severing of the blood vessels in the area. These procedures can even require the surgeon to cut through several blood vessels and ligate (tie) them to the trachea, in order to achieve an adequately large ostomy. The length of time needed to perform these procedures (often on the order of half an hour) is poorly suited to emergency treatment, when prompt restoration of the air passageway is critical. Moreover, the use of a scalpel to fully form an ostomy potentially causes undue trauma to the tissues surrounding the ostomy site, and can result in the formation of an unduly large or oversized opening in the soft tissue of the neck.

To minimize such trauma, it has been found desirable to initially incise only a small opening, and thereafter enlarge the opening with further dilation. For example, one prior technique for dilating an ostomy includes the use of a wire guide to facilitate the introduction of a dilator into the trachea. This technique involves the insertion of a needle and an over-the-needle catheter into the trachea. A drawback of this technique is that it requires the preliminary use of a scalpel to make an incision through the skin and cricothyroid membrane so that the needle can be inserted into the trachea. Even though intended to be performed in an emergency situation, the technique entails the sequential manipulation of several devices by the physician, which is time consuming and complicates the procedure.

Another procedure eliminates the use of the catheter and involves placing a wire guide through the needle itself. The ostomy formed by the needle is then dilated by the use of a device having a handle and a nose, the nose extending laterally from the axis of the handle. While this type of device offers more powerful dilation than is possible with elongated tubular dilators, a problem with this device is that the unguarded nose must be inserted into the trachea with precision, and must be manipulated at an angle, in order to avoid perforating the posterior tracheal wall.

Another prior art technique for dilating an ostomy is the use of a tapered, elongated, tubular dilator or a series of telescopically positionable, tapered dilators with increasingly larger diameters. A problem with these dilators is that each dilator presents a pointed distal end to the posterior tracheal wall when introduced into the trachea. The risk of injury to the trachea is compounded by the toughness of the tracheal membrane, which resists the introduction of medical devices.

An atraumatic device for forming and dilating an ostomy in a tracheal wall which avoids much of the trauma and damage to the tracheal wall encountered in many prior methods and devices is disclosed in U.S. Pat. No. 5,653,230, incorporated by reference herein. This device permits the enlargement of a formed ostomy without risk of perforating the rear of the trachea, and protects the cuff of a tracheal tube from damage during the insertion of the tracheal tube through the ostomy. The device employs a catheter having a polymeric inflatable balloon at the distal end of the catheter. A tip of a wire guide is inserted through the tracheal wall so that the wire guide lies across the tracheal wall. The catheter is positioned over the wire guide, and advanced until the balloon lies across the tracheal wall. The balloon is then inflated to atraumatically radially dilate a portion of the tracheal wall, thereby forming the dilated ostomy. Although this device is generally effective for atraumatically dilating the ostomy in the tracheal wall, the presence of sharp fragments of broken tracheal cartilage places the balloon at risk of puncture. In addition, balloons formed in this manner from polymers generally have a straight cylindrical orientation, and are resistant to bending and may have difficulty maintaining a bended orientation.

It would be desirable to provide an atraumatic dilation device for forming an ostomy in a tracheal wall that substantially avoids damage to the tracheal wall and permits the enlargement of a formed ostomy without risk of perforating the rear of the trachea. It would further be desirable to provide such a device that includes an inflatable member that is more resistant to breakage from tracheal cartilage fragments than prior art devices. It is still further desired to provide such an atraumatic device that can be inflated to have a curved orientation, thereby diminishing stress on the posterior wall of the trachea.

### BRIEF SUMMARY

The foregoing problems are solved and a technical advance is achieved in an illustrative device for forming an ostomy through the wall of a trachea. The device comprises a dilator tube and a reinforced balloon catheter carried by the dilator tube. During use, the dilator tube and the balloon catheter are advanced along a wire guide until the reinforced balloon lies across the tracheal wall. The balloon is inflated to atraumatically dilate a portion of the tracheal wall, thereby forming the desired dilated ostomy. The reinforced balloon catheter is resistant to sharp fragments, such as broken tracheal cartilage, that may otherwise place the balloon at increased risk of puncture. In addition, the reinforcement enables the balloon to be inflated in a curve, and to maintain the curved configuration during use, thereby diminishing the stress on the posterior wall of the trachea.

In one aspect, the present application is directed to a device for forming an ostomy in a tracheal wall. The device comprises a dilator tube having a distal end, and a reinforced balloon catheter carried by the dilator tube. The balloon catheter includes an inflatable balloon extending from the distal end of the dilator tube, wherein the dilator tube and the balloon catheter are advanceable along a wire guide percutaneously positionable across the tracheal wall. The balloon is adaptable to atraumatically dilate a portion of the tracheal wall to form an ostomy in the tracheal wall upon inflation of the balloon. An additional feature of the invention is that the reinforced balloon catheter may comprise coaxial inner and outer elastic tubes, wherein the inner tube includes a throughbore for the wire guide, and the inner and outer tubes define a balloon lumen therebetween. The reinforcement is preferably included in the outer elastic tube, and preferably comprises a metal or metal alloy reinforcement.

In another aspect, the present invention is directed to a percutaneous method of forming an ostomy in a tracheal wall. The method employs a reinforced balloon catheter adapted to atraumatically dilate a portion of the tracheal wall upon inflation of the balloon. The tip of a wire guide is percutaneously inserted through the tracheal wall so that the wire guide lies across the tracheal wall, and the reinforced balloon catheter is positioned over the wire guide. The balloon catheter is advanced along the wire guide until the balloon lies across the tracheal wall. The balloon is inflated while it lies across the tracheal wall to atraumatically dilate a portion of the tracheal wall and form an ostomy in the tracheal wall.

Further aspects and advantages of the invention are discussed below in conjunction with the preferred embodiments.

### BRIEF DESCRIPTION OF THE DRAWINGS

A better understanding of the present invention will now be had upon reference to the following detailed description, when read in conjunction with the accompanying drawing, wherein like reference characters refer to like parts throughout the several views, and in which:

FIG. 1 is a side view of a preferred embodiment of the dilational device of the present invention;

FIG. 2 is a side view of a portion of the balloon catheter of the device of FIG. 1;

FIG. 3 is a longitudinal sectional view of a portion of the balloon catheter;

FIG. 4 is a longitudinal view of a preferred embodiment of the balloon catheter showing a braided pattern of reinforcement wires, and illustrating the bendability of the balloon portion; and

FIG. 5 is an enlarged view of a portion of the balloon catheter illustrating a braiding pattern of the reinforcement wires.

### DETAILED DESCRIPTION OF THE DRAWINGS AND THE PRESENTLY PREFERRED EMBODIMENTS

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

The present invention relates to a dilational device having a reinforced balloon catheter. In the following discussion, the terms “proximal” and “distal” will be used to describe the opposing axial ends of the device, as well as the axial ends of various component features. The term “proximal” is used in its conventional sense to refer to the end of the device (or component thereof) that is closest to the operator during use of the device. The term “distal” is used in its conventional sense to refer to the end of the device (or component thereof) that is initially inserted into the patient, or that is closest to the patient.

FIG. 1 illustrates a preferred embodiment of a percutaneous dilational device 10 for forming an ostomy in a tracheal wall. The device 10 comprises a hollow dilator tube 12 having a tapered distal end 14, and a reinforced balloon catheter 16 coaxially carried by and disposed within a throughbore of the dilator tube 12.

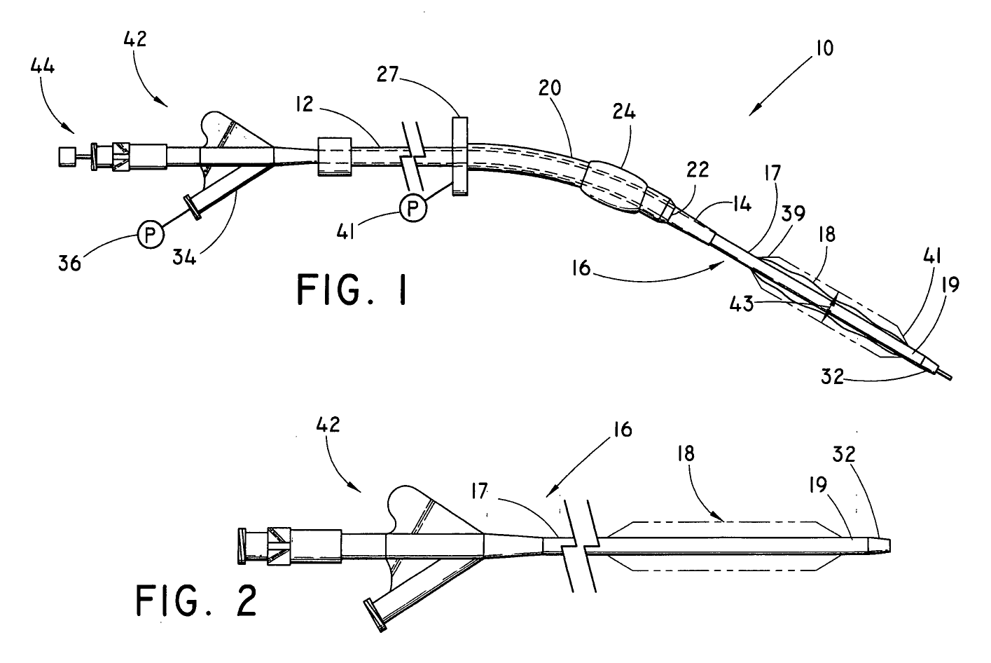

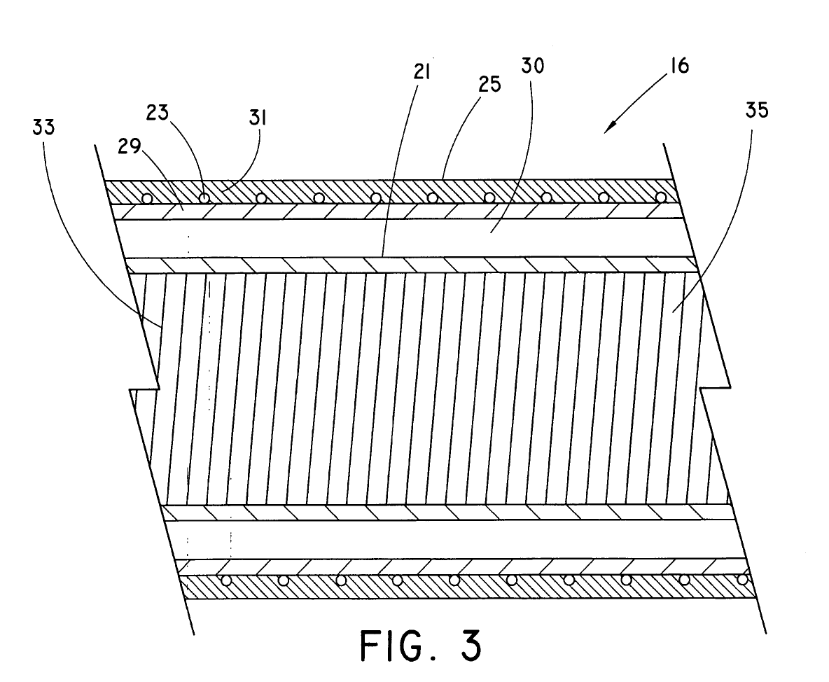

For clarity, the balloon catheter 16 is shown in FIG. 2 removed from the device. Balloon catheter 16 comprises a shaft portion 17, an inflatable balloon portion 18, and a distal tip portion 19. Preferably, distal tip 19 comprises an atraumatic tip. Balloon catheter 16 is a laminate structure comprising tightly-packed reinforcement wires 23, and a plurality of layers of a suitable polymeric material, such as polyurethane. A conventional manifold 42 may be provided at the proximal end of the catheter in well-known fashion.

The dilator tube 12 and the balloon catheter 16 are comprised of medical grade, synthetic materials. Balloon catheter 16 may be composed of a relatively flexible and slightly elastic material, while the dilator tube is preferably composed of a somewhat more rigid but somewhat resilient material. As shown in FIG. 1 in phantom, the balloon preferably has a generally cylindrical shape upon inflation, and includes tapered proximal end 39 and distal end 41. The inflated diameter of the balloon 18 (indicated by arrows 43) is selected in view of the size of the ostomy to be formed.

Preferably, the balloon catheter 16 is a coaxial system comprising an inner tube 21 and an outer tube 25, as shown in the fragmented view of FIG. 3. A balloon inflation lumen 30 is provided between respective inner and outer tubes 21 and 25, for supplying a fluid under pressure for inflating the balloon 18 in well-known fashion. The lumen 30 is open to a chamber 34 which fluidly connects the lumen 30 to a supply of pressurized fluid 36, the fluid supply 36 being conventional and indicated in only a general manner. In a preferred embodiment, the fluid provided by the fluid supply 36 is saline solution at about three bars pressure, which is adequate to atraumatically dilate a portion of a tracheal wall and thereby form an ostomy in the tracheal wall. A longitudinal throughbore 35 extends through the interior of inner tube 21.

A small plug of tubing 32 may be bonded to the distal end of catheter tip 19 to close the inflation lumen distally. Tubing plug 32 also has a longitudinal bore therethrough sized to enable passage of a wire guide 44. In the embodiment shown in FIG. 3, throughbore 35 is reinforced with a helical coil 33, preferably a flat steel coil coated with a suitable polymeric substance. The coil is preferably positioned on the interior of tube 21, and/or it can be extruded with tube 21. Coil 33 enhances the flexibility of the catheter and enables it to more easily bend. It also provides radial reinforcement to improve the crush resistance of the device.

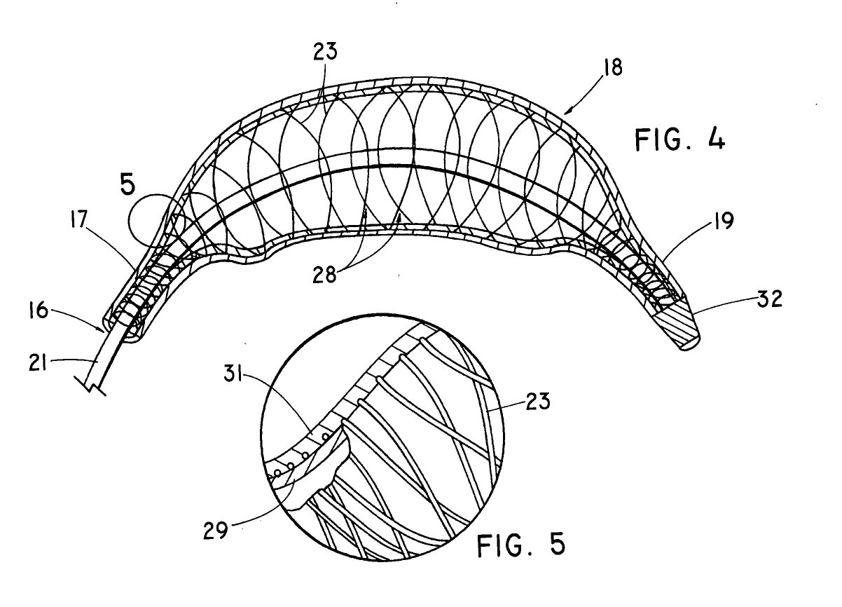

The inflatable balloon 18 is integrated in the structure of the outer tube 25. FIGS. 4 and 5 provide a more detailed illustration of a preferred embodiment of the inflatable balloon. In this embodiment, outer tube 25 comprises inner 29 and outer 31 tube layers. The reinforcement wires 23 are sandwiched in the inner 29 and outer 31 layers of the polymeric material. Preferably these layers are formed of a thermoplastic material such as polyurethane. In a preferred embodiment, the reinforcement wires 23 of the balloon catheter 16 comprise a braid consisting of, e.g., 48 wires which may be braided to a “stocking.”

The reinforcement wires 23 of each particular segment of the catheter (i.e., the shaft portion 17, inflatable balloon portion 18 and tip portion 19) extend circumferentially around the longitudinal axis of the balloon catheter, and are oriented at a specific angle (relative to the center axis), depending on the intended purpose of the particular catheter part e.g., whether during inflation strain the part is intended to expand or contract radially, shorten or lengthen axially, or be kept at the same dimension. The ratio between the change in the radial and axial dimensions is inversely proportional. Thus, if the catheter part contracts radially it simultaneously expands axially. Alternatively, if the catheter part expands radially it simultaneously contracts axially. When the angle of the braiding in a particular shaft part is smaller than the neutral angle (the angle that does not result in dimensional change during inflation strain), the catheter part contracts axially during inflation, i.e., it is shortened. When the angle of braiding is greater than the neutral angle, the catheter part is lengthened axially. In a preferred embodiment, the neutral angle \acute{α} is about 54.7°, although the device may be suitably constructed to have other neutral angles.

As it is not necessary to shorten or lengthen shaft 17, the angle of inclination of the wires in the shaft may in theory be the neutral angle (54.7°). However, in actuality, the neutral angle may be inappropriate at shaft portion 17 since the braiding at this angle may be so dense that it may be difficult to bond the inner and the outer polyurethane layers 29, 31 together between the wires of the reinforcement. This can be particularly significant in the proximal portion of shaft 17, where inner and outer tubes 29 and 31 are preferably melted together or otherwise merged in a manner to avoid moisture that may otherwise cause delamination during inflation. By making the braiding more loose (i.e., less dense) and the angle smaller than the neutral angle, the shaft will be slightly shortened during inflation.

Balloon 18 may be considered to be a “constructed weakness” in the braiding pattern of the distal portion of catheter outer tube 25. The balloon shape is formed as the catheter inflates radially during inflation strain. The angle of the braiding at this portion of balloon catheter 16 is smaller than the neutral angle, and the wires are arranged in a less dense manner than in the shaft portion. Consequently, the balloon part contracts axially and expands radially during inflation. When polyurethane is used in the catheter material, the balloon can stretch elastically in the radial direction up to about 500%. Thus, for example, a 7 Fr (2.3 mm) catheter can expand to create a balloon having a diameter of approximately 10 mm.

The braiding at the distal tip 19 is preferably somewhat more dense than that in the shaft 17, and the tip may therefore have a braiding angle that exceeds the neutral angle. In this event, tip 19 will be lengthened during compressive strain. This arrangement is beneficial because simultaneously with the lengthening, the tip crimps radially and fixes the bonding of the tip with the assembly.

Preferably, there is no special bond between the reinforcement wires so that the wires are movable with respect to each other at cross-over points 28 during expansion of the balloon. The cross-over points 28 are shown in FIG. 4. Although it is preferred that the filaments are arranged in a braided pattern as described, other conventional arrangements for reinforcement wires may alternatively be utilized, such as helical winding. The use of non-bonded reinforcement wires as described provides advantages not found in otherwise comparable balloon structures that are made up of, e.g., plastic reinforcements that do not include segments that are movable relative to each other. Most significantly, the use of the non-bonded wires allows the braid, and thus the balloon, to bend or flex when exposed to compressive stresses. As a result, the reinforcement braid and the balloon can conform to the shape of an internal passageway without permanent deformation of the wires.

Preferably, the reinforcement wires or filaments 23 are metallic, with stainless steel being most preferred. However, those skilled in the art will appreciate that the wires may be formed from other metals, alloys, and non-metallic compositions conventionally used in the medical device field for reinforcement purposes. One preferred metal alloy is the nickel/titanium alloy nitinol. Additional details of balloon catheters of a type that are suitable for use in the present invention are provided in U.S. Pat. No. 5,772,681, incorporated by reference herein.

The working pressure and the burst pressure of balloon catheter 16 depend largely on the strength of the wires of the braid, since the polyurethane does not contribute appreciable strength to the catheter. The strength of the reinforcement braid may be tested by subjecting the wires to heavy pulling during braiding. If any of the wires do not meet strength requirements, they will break during testing, and the catheter must be rejected. The pulling ensures that the catheters have a relatively homogeneous burst pressure and working pressure. Normally, to maintain a level of safety during use, the working pressure is maintained between about 15 and 20%, or more, below the burst pressure.

As stated, the inner and outer tubes of laminated balloon 18 are desirably composed of polyurethane. However, those skilled in that art will appreciate that other conventional medical grade flexible polymeric materials may alternatively be used, as well as medical grade vulcanized rubber compositions. Balloon 18 is constructed so that its diameter when inflated conforms to the size of ostomy to be formed. The length of the balloon is preferably about 60 mm long between its proximal and distal ends, although a balloon may be formed to have any length consistent with anatomical constraints in a particular patient.

The thin elastic supporting layer that forms the inner elastic tube 21 may be extruded or coated on a mandrel. This tube preferably has a thickness of about 0.05-0.10 mm.

The outer tube 25 (made up of layers 29 and 31, and reinforcement wires 23) also preferably has a wall thickness of about 0.05-0.10 mm. The reinforcement wires preferably have a diameter of about 0.02-0.04 mm. The dimensions provided above are examples only, and those skilled in the art will appreciate that the balloon catheter may be constructed having other dimensions.

As stated, during coating of the elastic outer layer, chemical or mechanical bonding between the cross-over points 28 of the reinforcement net should preferably be avoided. Compared to a helical laminate, the braided arrangement is considered more stable and its degree of flexibility larger during otherwise identical external pressurizing.

The purpose of forming an ostomy in the tracheal wall is to allow the insertion of a tracheal tube 20 through the tracheal wall, so as to establish an air passageway for the patient. The device 10 as described can be used for establishing an ostomy for the insertion of a separate tracheal tube 20 standing alone. However, the device will preferably include the tracheal tube 20 as well, coaxially carried on the dilator tube 12 adjacent to its distal end 14, as shown in FIG. 1.

The tracheal tube 20 may be formed of a conventional materials well-known in the art for such use, such as medical grade, substantially rigid synthetic material. Radiopaque polyvinyl chloride is one example of such material. The tracheal tube 20 possesses a permanent curve which facilitates its introduction into an ostomy in the tracheal wall, in a manner well known in the art. The tracheal tube 20 comprises a distal aperture 22 open to the trachea and lungs of the patient, and also comprises an inflatable circumferential cuff 24 positioned adjacent the distal aperture 22. As is conventional, the cuff 24 is desirably a thin wall, high volume, low pressure cuff, composed of a flexible and somewhat elastic material. This permits the cuff 24 to establish a good seal between the tracheal tube 20 and the trachea of the patient, cranial to the distal aperture 22 of the tracheal tube 20.

The tracheal tube 20 can further comprise a flange 27 for abutment against the skin of the patient when the tracheal tube 20 is inserted in the ostomy. The flange 27 is represented diagrammatically in the Figures as a flat disk, but can of course have other well-known configurations for flanges used in such devices. A supply 41 of low-pressure fluid (such as air) for inflating and deflating the cuff 24 is also represented diagrammatically in the Figures, and includes not only a fluid source or reservoir (not shown) but also any conventional tubes, bores or conduits employed to fluidly connect the cuff 24 to the fluid supply 41. The nature of such elements is well known and is believed not to be critical to the present invention. Therefore, these elements are not further described here.

The tracheal tube 20 possesses conventional dimensions suited to the patient into whom it will be introduced. For example, for adult patients, the tracheal tube 20 can typically have an outside diameter of about 8.5 to about 13.0 mm, and an inside diameter of about 5.0 to 9.0 mm. A tracheal tube 20 with an outside diameter of about 12.0 mm and an inside diameter of about 8.5 mm will be used as an example herein, merely by way of explanation, and not as a limitation of the invention.

The diameter of the dilator tube 12 and the diameter of the balloon 18 when inflated are selected to match the size of the tracheal tube 20 being inserted. For example, it is preferable but not essential that the outside diameter of the dilator tube 12 be very close to the inside diameter of the tracheal tube 20. Indeed, these two diameters can possess the same nominal value, that is, the dilator tube 12 can have the same nominal 8.5 mm diameter as the nominal 8.5 mm inside diameter of the tracheal tube 20. The slight resiliency of the dilator tube 12 permits this close tolerance; however, it may be advantageous to apply a water-soluble jelly or other lubricant over the dilator tube 12 to ensure that the tracheal tube 20 does not become stuck on the dilator tube 12.

It is also preferable that the balloon 18, when inflated, have a diameter equal to or slightly greater than the outside diameter of the tracheal tube 20 and uninflated cuff 24. For use with the tracheal tube 20 having an outside diameter of 12.0 mm, the balloon 18 should have a diameter when inflated of 12.0 mm, or slightly more, perhaps 0.5 to 1.0 mm more. This close sizing or slight oversizing of the balloon diameter as compared to the tracheal tube diameter ensures that the ostomy formed by the balloon 18 will be large enough to prevent damage to the uninflated cuff 24 during insertion of the tracheal tube 20 into the ostomy.

It should be noted that any separate tracheal tube 20 not carried by the dilator tube 12 can have the same features and construction as disclosed above. In such a case, the diameter of the balloon 18 when inflated should be only equal to, and not necessarily greater than, the outside diameter of the tracheal tube 20.

It is particularly advantageous, however, that the tracheal tube 20 be coaxially carried by the dilator tube 12. In such a case, the dilator tube 12, the balloon catheter 16 and the tracheal tube 20 are adapted for advancement along a wire guide 44 together, without longitudinal movement of any of them relative to one another during such advancement. Such movement as a single unit reduces the number of manipulative steps necessary to introduce the tracheal tube 20 into the ostomy, thereby making the introduction faster and easier to perform. The inserted tracheal tube 20 can be taped or strapped to the neck of the patient in the conventional manner.

It should be evident from the above discussion that the device 10 of the present invention can therefore comprise not only the combination of the dilator tube 12 and the balloon catheter 16, but optionally the tracheal tube 20. The device can also comprise a conventional wire guide, and if desired, a needle for introducing the device via the well-known Seldinger technique.

During placement of the device utilizing the Seldinger technique, the tip of the wire guide is initially inserted through the tracheal wall via a bore in the needle in well-known fashion. The dilational device 10 (which may or may not include a tracheostomy tube) is advanced along the wire guide until the uninflated balloon lies across the tracheal wall. The balloon is thereafter inflated while it lies across the tracheal wall to atraumatically dilate a portion of the tracheal wall and for an ostomy in the wall.

Any undisclosed details of the construction or composition of the various elements of the disclosed embodiment of the present invention are not believed to be critical to the achievement of the advantages of the present invention, so long as the elements possess the strength or flexibility needed for them to perform as disclosed. The selection of these and other details of construction are believed to be well within the ability of one of even rudimentary skills in this area, in view of the present disclosure.

It is to be understood that the above-described device is merely an illustrative embodiment of the principles of this invention, and that other devices and methods for using them may be devised by those skilled in the art, without departing from the spirit and scope of the invention. It is also to be understood that the invention is directed to embodiments both comprising and consisting of the disclosed parts and steps. It is therefore intended that the foregoing detailed description be regarded as illustrative rather than limiting, and that it be understood that it is the following claims, including all equivalents, that are intended to define the spirit and scope of this invention.

1. A device for forming an ostomy in a tracheal wall, said device comprising:
a dilator tube having a distal end; and
a reinforced balloon catheter carried by said dilator tube, the balloon catheter including an inflatable balloon extending from the distal end of the dilator tube, wherein the dilator tube and the reinforced balloon catheter are advanceable along a wire guide percutaneously positionable across the tracheal wall, and wherein the reinforced balloon is adapted to atraumatically dilate a portion of the tracheal wall to form an ostomy in the tracheal wall upon inflation of the balloon.

2. The device according to claim 1, wherein the balloon catheter is reinforced by a metal or a metal alloy.

3. The device according to claim 2, wherein the balloon reinforcement comprises stainless steel.

4. The device according to claim 2, wherein the balloon reinforcement comprises a nickel-titanium alloy.

5. The device according to claim 2, wherein the reinforced balloon catheter comprises an inner tube, and an outer elastic tube, said outer elastic tube comprising first and second layers, said reinforcement being disposed in at least one of said first and second layers .

6. The device according to claim 2, wherein the reinforced balloon catheter comprises coaxial inner and outer elastic tubes, said inner tube including a throughbore for said wire guide, and said inner and outer tubes defining a balloon lumen therebetween, the outer elastic tube including said reinforcement.

7. The device according to claim 6, where the reinforcement comprises a layer of reinforcing wire oriented in a braided pattern.

8. The device according to claim 7, wherein the reinforced balloon catheter further comprises a proximal shaft portion and a distal tip portion, and wherein each of said shaft portion, inflatable balloon and distal tip portion includes said reinforcement.

9. The device according to claim 8, wherein said reinforcement extends from the proximal end to the distal end of said balloon catheter.

10. The device according to claim 6, wherein said inner and outer tubes are formed from polymers.

11. The device according to claim 10, wherein said outer tube is formed from polyurethane.

12. The device according to claim 8, wherein said shaft portion axially contracts during inflation of said inflatable balloon.

13. The device according to claim 8, wherein said tip portion axially expands during inflation of said inflatable balloon.

14. The device according to claim 8, further comprising a tip bonded to the distal end of the balloon catheter for sealing said balloon lumen, said tip comprising a throughbore for said wire guide in registry with said inner tube throughbore.

15. The device according to claim 10, wherein said inflatable balloon portion is radially elastically stretchable upon inflation up to about 500% of a pre-inflation diameter.

16. The device according to claim 6, further comprising a tracheal tube carried by the dilator tube adjacent the distal end of the dilator tube.

17. The device according to claim 16, wherein the tracheal tube comprises a distal aperture and an inflatable circumferential cuff adjacent to the distal aperture, and wherein the balloon, when inflated, has a diameter equal to or greater than the diameter of the circumferential cuff of the tracheal tube when the circumferential cuff is uninflated.

18. The device according to claim 17, wherein the dilator tube, the balloon catheter and the tracheal tube are adapted for advancement along the wire guide together, without longitudinal movement of the dilator tube, the balloon catheter and the tracheal tube relative to one another during advancement.

19. The device of claim 1, wherein said reinforced balloon is inflatable in a curved orientation for diminishing stress on a posterior wall of the trachea upon insertion of the device.

20. A method of forming an ostomy in a tracheal wall employing a reinforced balloon catheter comprising an inflatable balloon, said balloon catheter adapted to atraumatically dilate a portion of the tracheal wall upon inflation of the balloon, said reinforcement comprising at least one of a metal and a metal alloy, the method comprising the steps of:
percutaneously inserting the tip of a wire guide through the tracheal wall so that the wire guide lies across the tracheal wall;
positioning the reinforced balloon catheter over the wire guide;
advancing the balloon catheter along the wire guide until the balloon lies across the tracheal wall; and
inflating the balloon while it lies across the tracheal wall to atraumatically dilate a portion of the tracheal wall and form an ostomy in the tracheal wall.

21. The method according to claim 19, wherein the reinforced balloon catheter further comprises coaxial inner and outer elastic tubes, said inner tube including a throughbore for said wire guide, said inner and outer tubes defining a balloon lumen therebetween, said outer elastic tube including said reinforcement.

22. The method according to claim 20, comprising the further steps of:
deflating the balloon; and
inserting a tracheal tube into the ostomy formed by inflation of the balloon.

23. The method according to claim 21, wherein the method is carried out with a dilator tube which carries the balloon catheter and the tracheal tube thereon, and wherein the inserting step comprises advancing the dilator tube, the balloon catheter and the tracheal tube together along the wire guide, without longitudinal movement of the dilator tube, the balloon catheter and the tracheal tube relative to one another.
